(19) United States
(12) Patent Application Publication
(10) Pub. No.: US 2021/0394140 A1
(43) Pub. Date: Dec. 23, 2021

(54) GAS MIXING DEVICE FOR LINEARIZING OR CALIBRATING GAS ANALYZERS

(71) Applicant: AVL LIST GMBH, GRAZ (AT)

(72) Inventors: MARIO ARAR, GRAZ (AT); HEINZ SCHIMPL, RAABA (AT); KARL NEUBAUER, LEIBNITZ (AT); ARESTIDE-VALENTIN DAMIEAN, FERNITZ (AT)

(73) Assignee: AVL LIST GMBH, GRAZ (AT)

(21) Appl. No.: 17/271,210

(22) PCT Filed: Aug. 28, 2019
(86) PCT No.: PCT/AT2019/060277

§ 371 Date: Feb. 25, 2021

(30) Foreign Application Priority Data
Aug. 28, 2018 (AT) A50736/2018

(51) Int. Cl. B01F 15/00; B01F 3/02; B01F 5/06; B01F 15/04; B01F 5/04

(52) U.S. Cl. CPC B01F 15/00344; B01F 3/028; B01F 5/0653; G01N 33/0006; B01F 15/0429; B01F 5/0473; B01F 15/00162

(57) ABSTRACT

A gas mixing device for linearizing or calibrating a gas analyzer. The gas mixing device includes a first gas inlet line for a first gas, a second gas inlet line for a second gas, a mixing channel having inlet openings which are arranged one behind the other in a flow direction, the inlet openings comprising a first upstream inlet opening and a second downstream inlet opening, and at least two valves which each comprise at least one inlet and one outlet. The at least two valves release or block a fluidic connection between at least one of the first gas inlet line and the second gas inlet line, and the mixing channel via the inlet openings. A flow cross section of the mixing channel is smaller at the first upstream inlet opening than at the second downstream inlet opening.

### CROSS REFERENCE TO PRIOR APPLICATIONS

This application is a U.S. National Phase application under 35 U.S.C. § 371 of International Application No. PCT/AT2019/060277, filed on Aug. 28, 2019 and which claims benefit to Austrian Patent Application No. A50736/2018, filed on Aug. 28, 2018. The International Application was published in German on Mar. 5, 2020 as WO 2020/041812 A1 under PCT Article 21(2).

### FIELD

The present invention relates to a gas mixing device for linearizing or calibrating gas analyzers, the gas mixing device comprising a first gas inlet line for a first gas, a second gas inlet line for a second gas, a mixing channel comprising at least two inlet openings that are arranged one behind the other in the flow direction, and at least two valves comprising at least one inlet and one outlet via which it is possible, via the at least two inlet openings, to release or block a fluidic connection between at least one of the gas inlet lines and the mixing channel.

### BACKGROUND

Gas mixing devices of this kind, which are also referred to as gas dividers, are highly precise devices via which exactly defined dilutions of calibration gases can be generated, which can then be provided to an analysis device for calibration, review and linearization.

These devices are known in particular from the field of exhaust gas analysis technology for motor vehicles. Supplying exactly defined dilutions is essential in this case since high percentage errors in the measurements would otherwise occur due to concentrations which are sometimes very small.

A gas mixing device of this kind is described, for example, in DE 30 00 949 A, where a device for generating a calibrating gas mixture is described with a mixing block comprising two gas inlet channels which are arranged on either side of a cylindrical mixing channel. Calibrating gas flows through the first channel, and zero gas or a carrier gas flows through the other. A connection between one of the inlet channels, in each case, and the mixing channel, can be interrupted or opened via valves. A sufficiently exact mixture as is required for measuring exhaust gas components cannot be achieved via this arrangement.

Critical nozzles are often used at the individual mixer stages in order to achieve these exact mixtures, through which critical nozzles, as of a certain input pressure, the same volume flow will always flow which is merely dependent on the smallest opening cross section and the temperature of the critical nozzle.

EP 0 690 985 B1 describes a gas mixing device in which four 3/2-way valves are connected in parallel with one another, which valves each comprise two inlets and one outlet, wherein a critical nozzle is arranged at the outlet. The smallest free cross section of these nozzles is in each case designed in the ratio 2:1 with respect to the following nozzle. It is thus possible for sixteen different mixing ratios to be generated at a high degree of accuracy from the calibration gas and the zero gas.

A problem of the known gas mixing devices is, however, that rinsing times are very long after performing a measurement using a calibration gas of a defined dilution since the cross section of the mixing channel must be set to the greatest possible volume flow, since the pressure drop in the mixing channel would otherwise be too great at a corresponding volume flow. Even an adjustment of the mixing channel cross section to a partial flow leads to inadmissibly high pressure losses.

This, however, results in only low flow rates existing at the start of the mixing tube where the full volume flow is not yet present, but rather only a partial flow, as a result of which a complete rinsing out takes a long time, in particular of the upstream regions. Although a corresponding adjustment of the mixing channel cross section to a partial flow would reduce flushing times, it would lead to inadmissibly high pressure losses in the downstream region.

### SUMMARY

An aspect of the present invention is to provide a gas mixing device for linearizing or calibrating gas analyzers, via which rinsing times can be significantly reduced while maintaining low pressure losses, so that the overall calibration time can be reduced.

In an embodiment, the present invention provides a gas mixing device for linearizing or calibrating a gas analyzer. The gas mixing device includes a first gas inlet line for a first gas, a second gas inlet line for a second gas, a mixing channel comprising at least two inlet openings which are arranged one behind the other in a flow direction, the at least two inlet openings comprising a first upstream inlet opening and a second downstream inlet opening, and at least two valves which each comprise at least one inlet and one outlet. The at least two valves are configured to release or to block a fluidic connection between at least one of the first gas inlet line and the second gas inlet line, and the mixing channel via the at least two inlet openings. A flow cross section of the mixing channel is smaller at the first upstream inlet opening than at the second downstream inlet opening.

### BRIEF DESCRIPTION OF THE DRAWINGS

The present invention is described in greater detail below on the basis of embodiments and of the drawings in which:

FIG. 1 shows a flow diagram of a gas mixing device according to the present invention for linearizing or calibrating a gas analyzer;

FIG. 2 shows a three-dimensional perspective view of an alternative gas mixing device according to the present invention;

FIG. 3 shows a three-dimensional perspective view of a detail of the gas mixing device according to the present invention from FIG. 2;

FIG. 4 shows a three-dimensional perspective view of a detail of the gas mixing device according to the present invention from FIG. 2, having a cross section through the part of a 3/2-way valve of the gas mixing device through which a flow passes; and

FIG. 5 shows a longitudinal section through a flow block of the gas mixing device according to the present invention according to FIGS. 2 and 3.

### DETAILED DESCRIPTION

Since the flow cross section of the mixing channel is smaller at a first upstream inlet opening than at a second downstream inlet opening, the cross section is adjusted to the volume flow present there. Due to the smaller cross section in the upstream region, in which the volume flow is also smaller, a higher velocity is thus achieved in the mixing channel, which leads to significantly reduced rinsing times.

In an embodiment of the present invention, the flow cross section of the mixing channel can, for example, widen continuously between the inlet openings in the flow direction. A pressure loss due to cross-sectional jumps is thereby reliably prevented.

Walls defining the mixing channel can, for example, be designed to extend continuously so as to reduce pressure losses over the entire length through which the flow travels and to achieve a uniform flow through the mixing channel.

In an embodiment of the present invention, the pressure loss between two successive inlet openings in the mixing channel can, for example, be equal to the pressure loss between two inlet openings successive in the downstream direction. This means that a constant pressure loss in each portion is provided over the entire mixing channel, so that constant measuring conditions are present and a uniform rinsing of the mixing channel occurs over the entire length thereof.

In an embodiment of the present invention, the flow cross section of the mixing channel can, for example, widen so that the flow rate directly downstream of one of the inlet openings in the mixing channel is equal to the flow rate directly downstream of the next inlet opening in the flow direction. The cross section of the mixing channel is designed so that a constant flow rate is present, for example, directly in front of or behind the inflow openings. As the volume flow increases, the cross section of the mixing channel is accordingly also increased in the same proportion.

The gas mixing device can, for example, comprise a plurality of 3/2-way valves which are connected in parallel, are arranged one behind the other in the flow direction, each of the plurality of 3/2-way valves comprising two inlets and one outlet, wherein, in a first switch position of the 3/2-way valve, a fluidic connection between the first gas inlet line and the mixing channel is established and, in a second switch position of the 3/2-way valve, a fluidic connection between the second gas inlet line and the mixing channel is established. Both gas inlet lines are thus controlled by the same valve so that one of the two gases always flows into the mixing channel via the inlet opening. These gases are typically a zero gas or a carrier gas, and a calibration gas of a known concentration. Each of the inlet openings is correspondingly controlled by a valve of this kind which can, for example, be produced as a permanently energized valve, as a result of which it is possible to also establish a thermal equilibrium in addition to the constant pressure ratios via the claimed design of the cross section.

It is furthermore advantageous for a critically operated nozzle to be arranged in the mixing channel, in a connection channel between the outlet of each valve, and the inlet opening. Constant volume flows can thereby be set with a high degree of accuracy since, as of a certain input pressure, an equal volume flow always passes through the nozzle and into the mixing channel, which flow is always only dependent on the smallest cross section and the temperature of the nozzle.

In an embodiment of the present invention, the critically operated nozzles downstream of the valves can, for example, be designed to have different narrowest cross sections, wherein the volume flow maximally achievable as a result of the narrowest cross section of each upstream nozzle corresponds to twice the volume flow maximally achievable as a result of the narrowest cross section of the following downstream nozzle, so that, for example, the narrowest cross section of each upstream nozzle corresponds to twice the cross section of the following downstream nozzle. This configuration makes it possible for a high number of different, clearly defined mixing ratios to be established, as a result of which a high number of sampling points for linearization or calibration are provided, which leads to very accurate measurement results in the subsequent operation of the gas analyzer. This also simplifies the design of the cross section for establishing a constant flow rate in the mixing channel.

The gas mixing device can, for example, comprise a flow block in which the two gas inlet lines and the mixing channel are formed, wherein a plurality of valves comprising downstream nozzles are fastened to the flow block, on either side of the flow block. The block-like design and the valves arranged on both sides increases the thermal stability of the block and thereby omits a plurality of assembly steps. When using 3/2-way solenoid valves which are energized in both end positions, it is even possible to achieve a constant temperature and thus a thermally stable state in the entire block following a heating time.

The gas inlet lines are advantageously arranged in the flow block so as to be in parallel with one another, on either side of the mixing channel, and the connection channels comprising the nozzles are arranged in the flow block so as to be in parallel with one another. This results in a unit that is very compact, easy to assemble, and easy to manufacture.

Successive inlet openings can, for example, be arranged oppositely on the mixing channel with respect to the central axis of the mixing channel. Better and quicker mixing of the two gases in the mixing channel are achieved thereby. The valves can also thereby be arranged at a shorter axial spacing from one another, which likewise leads to a reduction in the required installation space and a shortening of the mixing channel.

A gas mixing device for linearizing or calibrating gas analyzers is thus provided via which the rinsing times, and thus also the overall calibration time, can be reduced. The gas mixing device is additionally simple to assemble and manufacture, and requires less installation space. Constant pressure ratios are furthermore generated via which the measurement results during linearization or calibration are improved.

A non-limiting exemplary embodiment of a gas mixing device according to the present invention is shown in the drawings and will be described below under reference to the drawings.

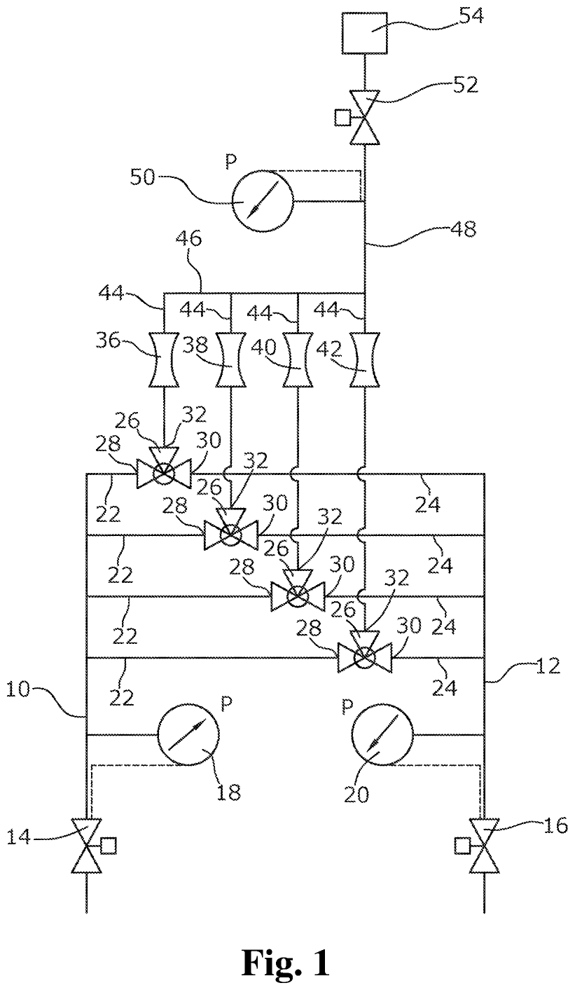

The gas mixing device shown in FIG. 1 consists of a first gas inlet line 10, which serves as a calibration gas supply line, and a second gas inlet line 12, which serves as a zero gas supply line. A control valve 14, 16 is arranged in each of the gas inlet lines 10, 12 in order to control a defined gas stream in the gas inlet lines 10, 12. A pressure sensor 18, 20 is arranged in each of the gas inlet lines 10, 12 downstream of the control valves 14, 16 for this purpose, via which pressure sensors 18, 20 the pressure in the gas inlet lines 10, 12 is measured, and a control unit is provided via which feedback to the control valves 14, 16, is achieved so that the pressure in the gas inlet lines 10, 12 can be regulated to a defined value.

Four gas supply lines 22, 24 branch off from each of the first gas inlet line 10 and the second gas inlet line 12, which gas supply lines 22, 24 each lead to a valve 26 that is designed as a 3/2-way valve, wherein each of the four 3/2-way valves 26 comprises two inlets 28, 30, where in each case the first inlet 28 is fluidically connected to the first gas inlet line 10 via one of the gas supply lines 22, and where in each case the second inlet 30 is fluidically connected to the second gas inlet line 12 via one of the gas supply lines 24. Each of the 3/2-way valves 26 comprises an outlet 32 through which, depending on the position of a sealing membrane 34 of the respective 3/2-way valve 26, either a zero gas stream or a calibration gas stream flows from the respective inlet 28, 30 via a critically operated nozzle 36 38, 40, 42 into a connection channel 44.

The critically operated nozzles 36, 38, 40, 42 are in each case arranged in a connection channel 44 and have different narrowest cross sections which are in each case graduated approximately at a ratio of 1:2, i.e., in the case of the four nozzles provided at a ratio of approximately 1:2:4:8. The next largest critically operated nozzle 36; 38; 40 is in each case located upstream of the following smaller nozzle 38; 40; 42. Since, as from a certain input pressure, which can be provided by the control valves 14, 16, together with the pressure sensors 18, 20, the same volume flow always flows through the critically operated nozzles 36, 38, 40, 42, which volume flow is dependent merely on the smallest opening cross section and the existing temperature of the relevant critically operated nozzle 36, 38, 40, 42, clearly defined volume flows of the carrier gas and of the calibration gas in a ratio of exactly 1:2 are thus generated at the different connection channels 44, downstream of the critically operated nozzles 36, 38, 40, 42. It is accordingly thereby possible to generate different defined mixing ratios between the two clean gas streams in that the positions of the 3/2-way valves 26 are changed accordingly.

For this purpose, the four connection channels 44 open in succession into a mixing channel 46, which in turn opens, downstream of the four mouths, into a gas outlet line 48 in which a pressure sensor 50 and a control valve 52 are likewise arranged. The gas outlet line 48 can be fluidically connected via the control valve 52 to a gas analyzer 54 which, in this way, can be provided with different mixing ratios for linearization or calibration, the evaluation results of which serve as sampling points for subsequent exhaust gas analysis of the gas analyzer 54.

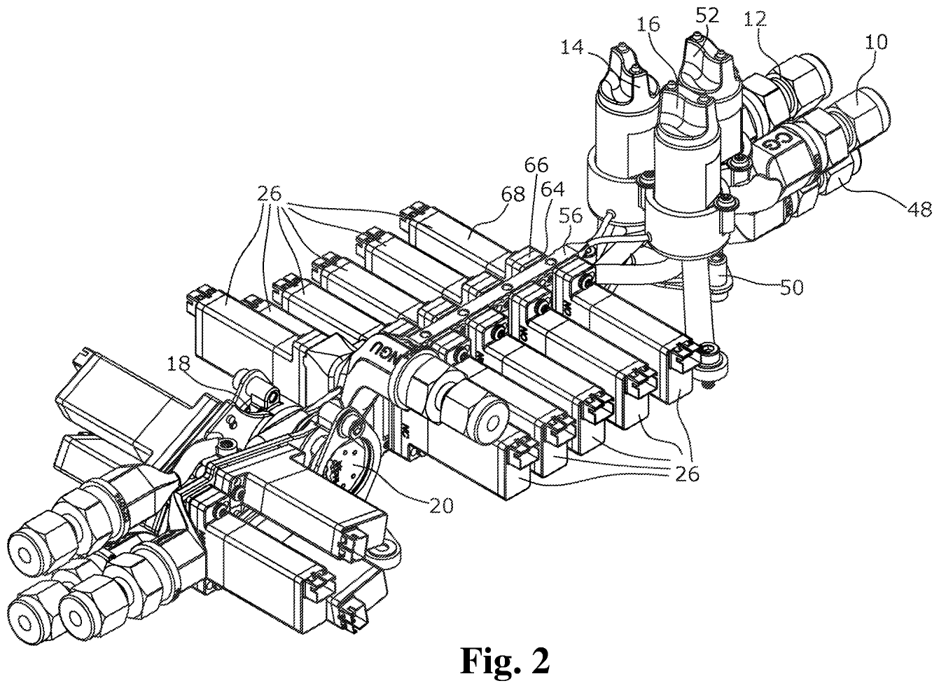

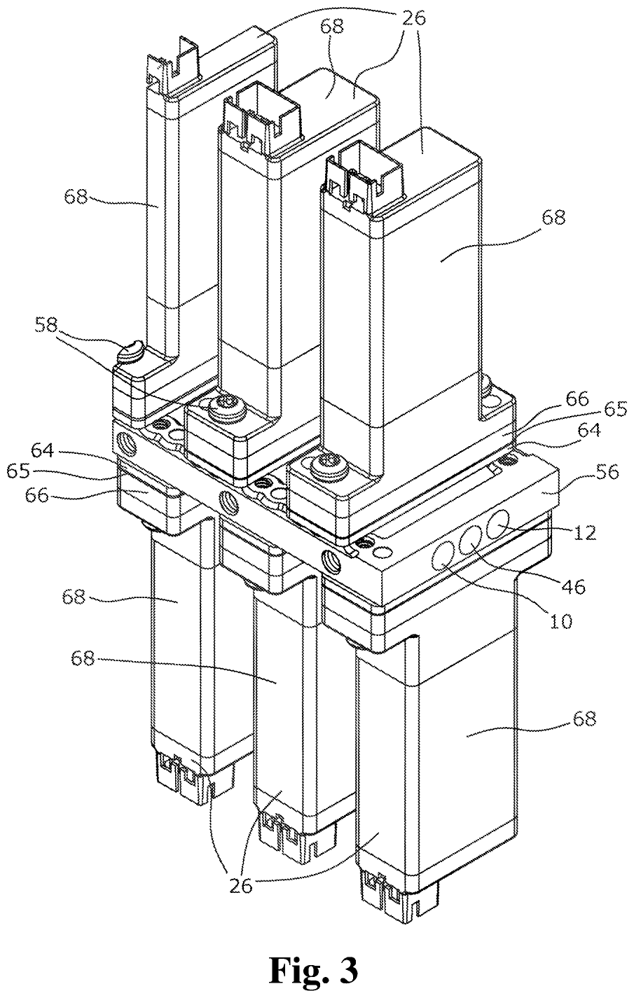

An embodiment for implementing this gas mixing concept is shown in FIGS. 2 to 4. In this case, the first gas inlet line 10, the second gas inlet line 12, at least in part, the gas supply lines 22, 24, the connection channels 44, and the mixing channel 46 are formed in a flow block 56, to which the 3/2-way valves 26 are fastened, on each side, via screws 58. The 3/2-way valves 26 are in this case fastened alternately on the two sides of the flow block 56, viewed in the axial direction of the mixing channel 46.

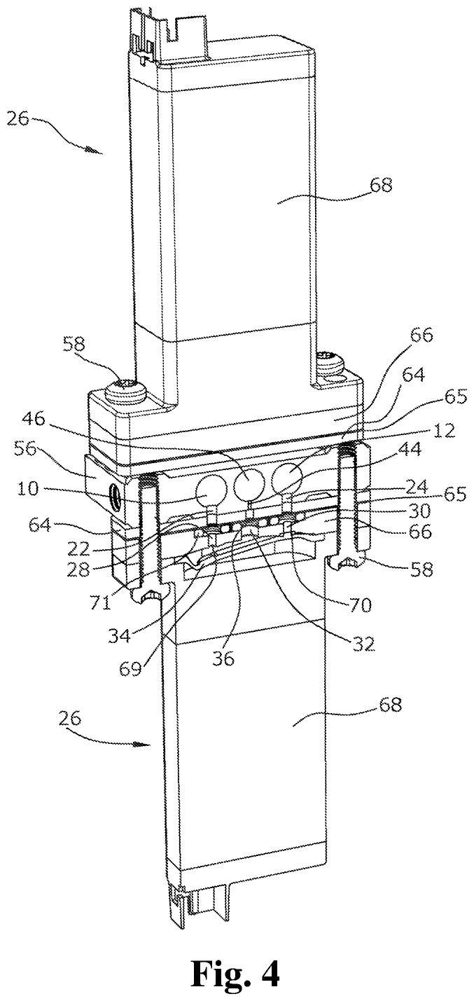

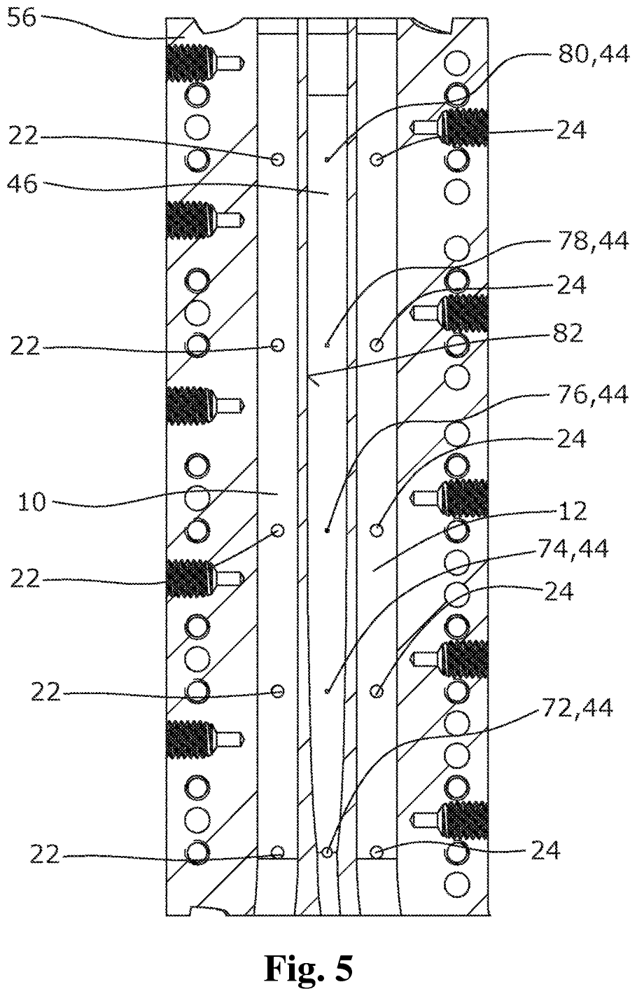

It can be seen in FIGS. 4 and 5 that, in the flow block 56, the mixing channel 46 is arranged between the two gas inlet lines 10, 12 and is aligned so as to be in parallel therewith. The gas supply lines 22, 24 and the connection channel 44 branch off from the gas inlet lines 10, 12 and the mixing channel 46 at an angle of 90°, and are likewise aligned so as to be parallel with one another. They are extended in a flat seal 64 which rests against a thin plate 65 in which the critically operated nozzles 36, 38, 40, 42 are formed and the opposite side of which in turn rests against a valve seat body 66 comprising two valve seats 69, 70 which surround the gas supply lines 22, 24 in this region and onto which the rocker-like sealing membrane 34 can be lowered, which sealing membrane 34, depending on the position thereof, rests either on the first valve seat 69 or on the second valve seat 70, and thus blocks either the calibration gas flow or the carrier or zero gas flow in the 3/2-way valve 26, or releases the other, respectively, to the connection channel 44. An electromagnetic actuator 68 for actuating the sealing membrane 34 is fastened to the valve seat body 66. The valve seat body 66 is sealed towards the outside, to the flat seal 64, via three O-rings 71 which surround the gas supply lines 22, 24 and which are interconnected by struts. The screws 58 for fastening accordingly penetrate the flat seal 64, the valve seat body 66, and the flange portion of the electromagnetic actuator 68, and are screwed in the flow block 56.

FIG. 5 shows each second gas supply line 22, 24 of the two gas inlet channels 10, 12, as well as inlet openings 72, 74, 76, 78, 80 in the central mixing channel 46, via which the connection channels 44 open into the mixing channel 46. According to the present invention, the flow cross section or the diameter of the mixing channel 46 increases from the first of the critically operated nozzles 36 or the largest first inlet opening 72, to the downstream second inlet opening 74. The widening of the flow cross section in the flow direction is formed between all the inlet openings 72, 74, 76, 78, 80.

The widening takes place continuously so that walls 82 defining the mixing channel 46 are also designed to extend continuously. The design of the widening of the mixing channel 46 is achieved in that a constant pressure loss between two successive inlet openings is in each case sought. This is achieved in that the flow cross section of the mixing channel 46 widens so that the flow rate in the mixing channel 46 is the same, in each case, directly downstream or directly upstream of the inlet openings 72, 74, 76, 78, 80, i.e., the cross sections are adjusted to the volume flow flowing in via the critically operated nozzles 36, 38, 40, 42, in each case. The cross section of the mixing channel 46 accordingly increases less and less in the flow direction since the volume flow supplied in each case halves according to the nozzle cross section which reduces in the flow direction of the mixing channel 46. A continuous widening is, however, selected in order to avoid cross-sectional jumps and associated turbulence, which would lead to increase pressure losses.

It is also noted that, in the cross section according to FIG. 5, only every other inlet opening 72, 74, 76, 78, 80 is visible but, in order to achieve the selected flow rate, the widening of the mixing channel 46 from each individual visible inlet opening 72, 74, 76, 78, 80 to the next inlet opening, not visible in FIG. 5, i.e., the radially opposite inlet opening with respect to the central axis of the mixing channel 46, is to be designed correspondingly.

This configuration of the mixing channel makes it possible to significantly reduce the rinsing time between the calibration measurements since the flow rate at the start of the mixing channel is significantly increased by the reduced cross-sectional area compared with known embodiments, and thus the calibration gas previously present in the mixing channel reaches the gas outlet line more quickly than was the case in known designs in which the cross section of the mixing channel was designed for the volume flow at the end of the mixing channel, in order to prevent pressure losses, in operation, that are too high. Reducing the rinsing times also reduces the dwell time of the calibration gas and thus the measuring time during the linearization or calibration as well as, consequently, the overall calibration time. In the case of the linearization or calibration, largely constant pressure ratios additionally occur via which the measurement results during linearization or calibration are improved. The present gas mixing device is additionally very compact, robust, and simple to assemble.

It should be clear that the scope of protection of the present main claim is not limited to the embodiments described. Depending on the configuration, it is in particular possible that a uniform widening of the mixing channel may not be selected, but rather a continuous but more quickly increasing channel widening may be provided in the region of the inlet opening. It is also possible for other valves to be used or for individual channels to be mounted, instead of the flow block. Further modifications within the scope of protection are also conceivable. Reference should also be had to the appended claims.

### LIST OF REFERENCE NUMERALS

10 First gas inlet line
12 Second gas inlet line
14 Control valve
16 Control valve
18 Pressure sensor
20 Pressure sensor
22 Gas supply line
24 Gas supply line
26 3/2-way valve
28 First inlet
30 Second inlet
32 Outlet
34 Sealing membrane
36 Critically operated nozzle
38 Critically operated nozzle
40 Critically operated nozzle
42 Critically operated nozzle
44 Connection channel
46 Mixing channel
48 Gas outlet line
50 Pressure sensor
52 Control valve
54 Gas analyzer
56 Flow block
58 Screws
64 Flat seal
65 Thin plate
66 Valve seat body
68 Electromagnetic actuator
69 First valve seat
70 Second valve seat
71 O-rings
72 First inlet opening
74 Second inlet opening
76 Inlet opening
78 Inlet opening
80 Inlet opening
82 Walls

What is claimed is:

1-11. (canceled)

12. A gas mixing device for linearizing or calibrating a gas analyzer, the gas mixing device comprising:
a first gas inlet line for a first gas;
a second gas inlet line for a second gas;
a mixing channel comprising at least two inlet openings which are arranged one behind the other in a flow direction, the at least two inlet openings comprising a first upstream inlet opening and a second downstream inlet opening; and
at least two valves which each comprise at least one inlet and one outlet, the at least two valves being configured to release or to block a fluidic connection between at least one of the first gas inlet line and the second gas inlet line, and the mixing channel via the at least two inlet openings,
wherein,
a flow cross section of the mixing channel is smaller at the first upstream inlet opening than at the second downstream inlet opening.

13. The gas mixing device as recited in claim 12, wherein the flow cross section of the mixing channel widens continuously between the at least two inlet openings in the flow direction.

14. The gas mixing device as recited in claim 12, wherein the mixing channel is defined by walls which are arranged to extend continuously.

15. The gas mixing device as recited in claim 12, wherein a pressure loss between two successive inlet openings of the at least two inlet openings is equal to a pressure loss between two inlet openings of the at least two inlet openings which are successive in a downstream direction.

16. The gas mixing device as recited in claim 12, wherein the flow cross section of the mixing channel widens so that a flow rate directly downstream of one of the at least two inlet openings in the mixing channel is equal to a flow rate directly downstream of the next one of the at least two inlet openings in the flow direction.

17. The gas mixing device as recited in claim 12, wherein,
each of the at least two valves is configured as a 3/2-way valve,
the at least two valves are connected in parallel and are arranged one behind the other in the flow direction, and
each of the at least two valves comprise two inlets and one outlet so that, in a first switch position of each of the at least two valves, a fluidic connection between the first gas inlet line and the mixing channel is established, and, in a second switch position of each of the at least two valves, a fluidic connection between the second gas inlet line and the mixing channel is established.

18. The gas mixing device as recited in claim 17, further comprising:
a connection channel arranged between the one outlet of each of the at least two valves and each the at least two inlet openings in the mixing channel; and
a critically operated nozzle arranged in each respective connection channel.

19. The gas mixing device as recited in claim 18, wherein,
each critically operated nozzle arranged downstream of the at least two valves is configured to have a different narrowest cross section, and
a volume flow maximally achievable as a result of a narrowest cross section of each critically operated nozzle which is upstream corresponds to twice a volume flow maximally achievable as a result of a narrowest cross section of the critically operated nozzle which follows downstream.

20. The gas mixing device as recited in claim 19, further comprising:
a flow block in which is formed the first gas inlet line, the second gas inlet line, and the mixing channel,
wherein,
the at least two valves comprising the respective critically operated nozzles arranged downstream are fastened to the flow block on either side of the flow block.

21. The gas mixing device as recited in claim 20, wherein,
the first gas inlet line and the second gas inlet line are arranged in the flow block so as to be parallel with each other on either side of the mixing channel, and
each connection channel comprising the critically operated nozzle is arranged in the flow block so as to be in parallel with one another.

22. The gas mixing device as recited in claim 12, wherein,
the mixing channel comprises a central axis, and
successive of the at least two inlet openings are arranged oppositely, on the mixing channel, with respect to the central axis of the mixing channel.
